(19) United States
(12) Patent Application Publication
(10) Pub. No.: US 2022/0154138 A1
(43) Pub. Date: May 19, 2022

(54) METHOD OF PRODUCING PLACENTA-LIKE ORGANOID, PLACENTA-LIKE ORGANOID, AND PRODUCTION OR TEST KIT

(71) Applicant: FUJIFILM Corporation, Tokyo (JP)

(72) Inventors: Koji MURAYA, Ashigarakami-gun (JP); Hidenori AKUTSU, Tokyo (JP); Hiroyuki NAKAMURA, Tokyo (JP); Tomoyuki KAWASAKI, Tokyo (JP)

(73) Assignee: FUJIFILM Corporation, Tokyo (JP)

(21) Appl. No.: 17/530,855

(22) Filed: Nov. 19, 2021

(30) Foreign Application Priority Data
Nov. 19, 2020 (JP) 2020-192182
Mar. 23, 2021 (JP) 2021-048452

(51) Int. Cl. C12N 5/073

(52) U.S. Cl. CPC C12N 5/0605; C12N 2501/115; C12N 2501/155

(57) ABSTRACT

There are provided a method of producing a placenta-like organoid that can be subjected to long-term culture, a placenta-like organoid that is produced by the above producing method, and a production or test kit that contains the above placenta-like organoid.

The method of producing a placenta-like organoid includes subjecting a pluripotent stem cell to suspension culture in the presence of a bone morphogenetic protein BMP4.

### CROSS-REFERENCE TO RELATED APPLICATIONS

This application claims priority under 35 U.S.C 119 to Japanese Patent Applications No. 2020-192182 filed on Nov. 19, 2020 and No. 2021-048452 filed on Mar. 23, 2021. Each of the above applications is hereby expressly incorporated by reference, in its entirety, into the present application.

### BACKGROUND OF THE INVENTION

### 1. Field of the Invention

The present invention relates to a method of producing a placenta-like organoid from a pluripotent stem cell. The present invention further relates to a placenta-like organoid that is produced by the above method, and a production or test kit.

### 2. Description of the Related Art

The placenta is an important organ for fetal development in the uterus. The placenta connects a fetus with the maternal environment through the umbilical cord, exchanges gas, nutrients, and excrement, and further, supports the production of pregnancy-related hormones and the immune defense of the fetus. The placenta is composed of cells of the trophoblastic lineage.

In STEM CELLS AND DEVELOPMENT, S. A. Sudheer et. al, Volume 21, Number 16, 2012, 2987-3000, it is described that in a case where a human embryonic stem cell (a human ES cell) is induced to differentiate into a syncytiotrophoblast (a kind of cell constituting the placenta, which produces human chorionic gonadotropin (hCG)) using bone morphogenetic protein-4 (BMP4), the differentiation is efficiently performed by inhibiting the fibroblast growth factor (FGF) signal transduction pathway. In STEM CELLS AND DEVELOPMENT, S. A. Sudheer et. al, Volume 21, Number 16, 2012, 2987-3000, cell culture is performed on a plate coated with Matrigel.

WO2016/186078A1 describes that a human pluripotent stem cell is subjected to adhesion culture in a medium containing a BMP signal transduction activating substance such as BMP4, and the cell on culture is brought into contact with the BMP signal activating substance to obtain a culture differentiated into the trophoblast.

JP2005-520514A describes that a primate stem cell is cultured in a medium, to which 1 to 100 ng/mL of a protein trophoblast-inducing factor such as BMP4, BMP2, BMP7, or growth/differentiation factor 5 (GDF5) is added, to obtain the human trophoblast.

JP2016-214138A describes placental stem cells are dispersed in aggregates of embryonic stem cells and are subjected to suspension culture in a medium containing FGF, a Wnt signal inhibitor, and the like to obtain the trophoblastic ectoderm-like vesicular structure body.

### SUMMARY OF THE INVENTION

Substances ingested by a pregnant woman permeate the placenta, and a fetus is exposed to the permeated substances. Whether or not a substance permeates the placenta greatly affects the exhibition of developmental toxicity to the fetus due to the substance, and thus an in vitro experimental system for evaluating an influence of a substance on the human placental function and the placental permeability of the substance is required to be developed. However, in a case where cells were induced to differentiate into trophectoderm and further a trophoblast lineage by two-dimensional adhesion culture, hCG production stopped in around two weeks, and thus long-term culture was difficult.

An object of the present invention is to provide a method of producing a placenta-like organoid that can be subjected to long-term culture. Further, another object of the present invention is to provide a placenta-like organoid that is produced by the above-described producing method and a production or test kit containing the above-described placenta-like organoid.

As a result of diligent studies to solve the above problems, the inventors of the present invention have found that in a case where a pluripotent stem cell is subjected to suspension culture in the presence of a bone morphogenetic protein BMP4, a placenta-like organoid capable of being subjected to long-term culture can be produced. The present invention has been completed based on the above findings. According to the present invention, the following inventions are provided.

<1> A method of producing a placenta-like organoid, comprising subjecting a pluripotent stem cell to suspension culture in a presence of a bone morphogenetic protein BMP4.

<2> The method according to <1>, further comprising bringing the pluripotent stem cell into a spheroid state.

<3> The method according to <2>, further comprising subjecting the pluripotent stem cell in the spheroid state to suspension culture in a presence of the bone morphogenetic protein BMP4 after the pluripotent stem cell is brought into the spheroid state.

<4> The method according to <2> or <3>, in which the bringing of the pluripotent stem cell into the spheroid state is carried out in a presence of basic fibroblast growth factor bFGF.

<5> The method according to any one of <2> to <4>, in which the bringing of the pluripotent stem cell into the spheroid state is carried out in an absence of the bone morphogenetic protein BMP4.

<6> The method according to any one of <1> to <5>, in which the subjecting of the pluripotent stem cell to the suspension culture in the presence of the bone morphogenetic protein BMP4 is carried out in a presence of basic fibroblast growth factor bFGF.

<7> The method according to any one of <1> to <6>, in which at least one of the bringing of the pluripotent stem cell into a spheroid state or the subjecting of the pluripotent stem cell to the suspension culture in the presence of the bone morphogenetic protein BMP4 is carried out in a presence of a microcarrier.

<8> The method according to <7>, in which the microcarrier is porous.

<9> The method according to any one of <1> to <8>, in which the pluripotent stem cell is an embryonic stem cell, an embryonic germ cell, or an induced pluripotent stem cell.

<10> The method according to any one of <1> to <9>, in which the placenta-like organoid is capable of producing at least one selected from the group consisting of chorionic gonadotropin, estradiol, dehydroepiandrosterone, 11-deoxycorticosterone, progesterone, pregnenolone, and allopregnanolone.

<11> A placenta-like organoid that is produced by the method according to any one of <1> to <9>.

<12> A production or test kit comprising the placenta-like organoid according to <11>.

<13> The production or test kit according to <12>, in which the kit is used for producing a reproductive hormone, evaluating toxicity or safety of a test substance, or analyzing an infection mechanism of a pathogen.

A placenta-like organoid that is produced by a producing method according to an aspect of the present invention can continue to produce hCG for more than 2 weeks and can be subjected to long-term culture.

### BRIEF DESCRIPTION OF THE DRAWINGS

FIG. 1 shows spheroids formed in a spinner flask on the 4th day of culture.

FIG. 2 shows changes in hCG production amount in the two-dimensional culture and the three-dimensional culture.

FIG. 3 shows the formation of a placenta-like organoid by the three-dimensional suspension cultures using a level 1 (using porous microspheres), a level 3 (using microcarriers), and a level 4 (without microcarriers).

FIG. 4 shows the positioning of a steroid hormone that is produced by the placenta.

FIG. 5 shows results of comparing the appearances of placental-like organoids before infection and on the 4th day after infection in a case where the placenta-like organoid has been infected with human cytomegalovirus (HCMV) AD-169 strain.

FIG. 6 shows results of checking, by electrophoresis, mRNA of an immediate-early 2 (IE2) gene of which the expression is increased in the early stage of viral infection, on the 1st day after infection (1 day post infection (dpi)) and the 5th day after infection (5 dpi), in a case where the placenta-like organoid is infected with human cytomegalovirus (HCMV) AD-169 strain.

### DESCRIPTION OF THE PREFERRED EMBODIMENTS

Hereinafter, embodiments for carrying out the present invention will be described in detail.

A method of producing a placenta-like organoid according to the embodiment of the present invention includes subjecting a pluripotent stem cell to suspension culture in a presence of a bone morphogenetic protein BMP4.

According to the configuration of the present invention, cells induced to differentiate by three-dimensional suspension culture continue to produce hCG for more than two weeks, whereas cells induced to differentiate by two-dimensional adhesion culture stop producing hCG in around two weeks. It has been confirmed that hCG production is continued for 2 months or longer, and it is possible to construct a production system of a placental hormone such as this hCG. In addition, by utilizing characteristics of a placental-like organoid that can be subjected to long-term culture, it is possible to evaluate what substance among foods and pharmaceutical products ingested by a pregnant woman affects placental function, or what substance passes through the placenta and how such a substance affects the fetal development or fetal development of the central nervous system and other tissues. Furthermore, it is possible to analyze the mechanism of an infectious disease caused by a virus with which a fetus is infected from a mother via the placenta.

The present invention provides a method of inducing differentiation from a pluripotent stem cell such as an embryonic stem cell (an ES cell), an embryonic germ cell (an EG cell), or an induced pluripotent stem cell (an iPS cell) to a cell or tissue of the trophectoderm lineage including the placenta. In the related art, differentiation induction has been carried out by two-dimensional adhesion culture on a plate of which the surface has been coated with laminin or Matrigel; however, in that case, the production of hCG, which is a placenta-specific hormone, stops in around two weeks. In addition, in an example of the related art, the differentiation induction is performed using a culture plate coated in advance with laminin (iMatrix-511: Nippi. Inc.). That is, 2 mL of an iPS cell medium (StemFit (registered trade name) AK02N: Ajinomoto Co., Inc.) is dispensed into a 6-well plate coated with laminin, BMP4 is added thereto, and then iPS cells (around 4×104 cells/well) are seeded. A placental hormone hCG begins to be produced around the 7th day of culture, but the hCG production stops around the 14th day. In some cases, cells begin to detach from the plate around the culture stage thereof, and the culture cannot be continued.

On the other hand, in the present invention, differentiation induction is carried out by carrying out three-dimensional suspension culture with stirring culture using a spinner flask or the like or stationary culture using a U-shaped bottom 96-well plate for spheroid preparation or the like. As a result, a placenta-like organoid that can continue to produce hCG for a long period of time is obtained. This organoid can be cultured for a long period of 2 months or longer.

Pluripotent Stem Cell

“Pluripotent stem cell” refers to a cell having both the ability (the differentiation pluripotency) to differentiate into all cells that constitute a living body and the ability (the self-replication ability) to generate daughter cells having the same differentiation potency as the mother cell through cell division. The differentiation pluripotency can be evaluated by transplanting an evaluation target cell into a nude mouse and testing for the presence or absence of formation of teratoma that includes cells of the respective three germ layers (ectoderm, mesoderm, and endoderm).

Examples of the pluripotent stem cell include an embryonic stem cell (an ES cell), an embryonic germ cell (an EG cell), and an induced pluripotent stem cell (an iPS cell); however, examples thereof are not limited thereto as long as a cell has both differentiation pluripotency and self-replication ability. An ES cell or an iPS cell is preferably used. An iPS cell is more preferably used. The pluripotent stem cell is preferably a mammalian (for example, primates such as a human or a chimpanzee, rodents such as a mouse or a rat) cell. And particularly preferably a human cell. Accordingly, in a preferred embodiment of the present invention, a human iPS cell or a human ES cell is used as the pluripotent stem cell, and in the most preferred embodiment, a human iPS cell is used.

The ES cell can be established, for example, by culturing an early embryo before implantation, an inner cell mass constituting the above early embryo, or a single blastomere (Manipulating the Mouse Embryo, A Laboratory Manual, Second Edition, Cold Spring Harbor Laboratory Press (1994); Thomason, J. A. et al., Science, 282, 1145-1147 (1998)). As the early embryo, an early embryo prepared by nuclear transfer of a somatic cell nucleus may be used (Wilmut et al. (Nature, 385, 810 (1997)), Cibelli et al. (Science, 280, 1256) (1998)), Akira Iriya et al. (Protein, nucleic acid and enzyme, 44, 892 (1999)), Baguisi et al. (Nature Biotechnology, 17, 456 (1999)), Wakayama et al. (Nature, 394, 369 (1998)); Nature Genetics, 22, 127 (1999); Proc. Natl. Acad. Sci. USA, 96, 14984 (1999)), Rideout III et al. (Nature Genetics, 24, 109 (2000), Tachibana et al. (Human Embryonic Stem Cells Delivered by Somatic Cell Nuclear Transfer, Cell (2013) in press). As the early embryo, a parthenogenetic embryo may be used (Kim et al. (Science, 315, 482-486 (2007)), Nakajima et al. (Stem Cells, 25, 983-985 (2007)), Kim. et al. (Cell Stem Cell, 1,346-1,352 (2007)), Revazova et al. (Cloning Stem Cells, 9, 432-449 (2007)), Revazova et al. (Cloning Stem Cells, 10, 11-24 (2008)). In addition to the above-described papers, regarding the preparation of an ES cell, the following can be referenced, Strelchenko N. et al. Reprod Biomed Online. 9: 623-629, 2004; Klimanskaya I., et al. Nature 444: 481-485, 2006; Chung Y., et al. Cell Stem Cell 2: 113-117,2008; Zhang X., et al. Stem Cells 24: 2669-2676, 2006; Wassarman, P. M. et al. Methods in Energy, Vol. 365, 2003, and the like. In addition, a fused ES cell obtained by cell fusion of an ES cell with a somatic cell is also included in the embryonic stem cell that is used in the method according to the embodiment of the present invention.

Some ES cells are available from conservation institutions or are commercially available. For example, human ES cells are available from National Research Institute for Child Health and Development (for example, SEES1-7), Institute for Frontier Medical Sciences, Kyoto University (for example, KhES-1, KhES-2, and KhES-3), WiCell Research Institute, and ESI BIO.

The EG cell can be established by, for example, culturing a primordial germ cell in the presence of a leukemia inhibitory factor (LIF), a basic fibroblast growth factor (bFGF), and a stem cell factor (SCF) (Matsui et al., Cell, 70, 841-847 (1992), Shamblott et al., Proc. Natl. Acad. Sci. USA, 95 (23), 13726-13731 (1998), Turnpenny et al., Stem Cells, 21 (5), 598-609, (2003)).

“Induced pluripotent stem cell (iPS cell)” is a cell having pluripotency (multiple differentiation potency) and proliferation ability, which is prepared by reprogramming a somatic cell by introducing reprogramming factors or the like. The induced pluripotent stem cell exhibits properties similar to the ES cell. The somatic cell that is used for preparing an iPS cell is not particularly limited and may be a differentiated somatic cell or an undifferentiated stem cell. In addition, the origin of the somatic cell is not particularly limited: however, a somatic cell of a mammal (for example, primates such as a human or a chimpanzee, rodents such as a mouse or a rat) cell is preferably used, and a human cell particularly preferably used. The iPS cell can be prepared by various methods reported so far. In addition, it is naturally expected that an iPS cell preparing method to be developed in the future will be applied.

The most basic method of preparing an iPS cell is a method of introducing four transcription factors, Oct3/4, Sox2, Klf4, and c-Myc, into a cell using a virus (Takahashi K, Yamanaka S: Cell 126 (4), 663-676, 2006; Takahashi, K, et al: Cell 131 (5), 861-72, 2007). It has been reported that human iPS cells have been established by introducing four factors, Oct4, Sox2, Lin28, and Nanog (Yu J, et al.: Science 318 (5858), 1917-1920, 2007). It has also been reported that iPS cells are established by introducing three factors excluding c-Myc (Nakagawa M, et al: Nat. Biotechnol. 26 (1), 101-106, 2008), two factors of Oct3/4 and Klf4 (Kim J B, et al: Nature 454 (7204), 646-650, 2008), or only Oct3/4 (Kim J B, et al: Cell 136 (3), 411-419, 2009). In addition, an establishing method of introducing a protein, which is an expression product of a gene, into a cell (Zhou H, Wu S, Joo J Y, et al: Cell Stem Cell 4, 381-384,2009; Kim D, Kim C H, Moon J I, et al.: Cell Stem Cell 4, 472-476, 2009) has also been reported. On the other hand, it has been also reported that it is possible to improve the preparation efficiency or reduce the factors to be introduced, by using an inhibitor BIX-01294 for a histone methyltransferase G9a, a histone deacetylase inhibitor valproic acid (VPA), Bay K8644, or the like (Huangfu D, et al: Nat. Biotechnol. 26 (7), 795-797, 2008; Huangfu D, et al: Nat. Biotechnol. 26 (11), 1269-1275, 2008; Silva J., et al: PLoS. Biol. 6 (10), e253, 2008). In addition, gene introducing methods have been studied as well, and techniques using, in addition to retroviruses, the following substances have been developed; lentiviruses (Yu J, et al: Science 318 (5858), 1917-1920, 2007), adenoviruses (Stadtfeld M, et al: Science 322 (5903), 945-949, 2008), plasmids (Okita K, et al: Science 322 (5903), 949-953, 2008), transposon vectors (Woltjen K, Michael I P, Mohseni P, et al: Nature 458, 766-770, 2009; Kaji K, Norrby K, Pac a A, et al: Nature 458, 771-775, 2009; Yusa K, Rad R, Takeda J, et al: Nat Methods 6,363-369, 2009), or episomal vectors (Yu J, Hu K, Smuga-Otto K, Tian S, et al: Science 324, 797-801, 2009).

Cells transformed to iPS cells, that is, cells that have undergone initialization (reprogramming) can be selected using, as an index, the expression of pluripotent stem cell markers (undifferentiated markers) such as Fbxo15, Nanog, Oct4, Fgf-4, Esg-1, and Cript, or the like. The selected cells are collected as the iPS cell.

iPS cells can be available, for example, from FUJIFILM Cellular Dynamics, Inc.; National University Corporation, Kyoto University; or Independent Administrative Institution, Institute of Physical and Chemical Research, BioResource Research Center.

Subjecting pluripotent stem cell to suspension culture in presence of bone morphogenetic protein BMP4

The method of producing a placenta-like organoid according to the embodiment of the present invention involves subjecting a pluripotent stem cell to suspension culture in the presence of a bone morphogenetic protein BMP4. In addition, the period for culturing the pluripotent stem cell in the presence of BMP4 is not particularly limited; however, it is preferably 2 to 20 days and more preferably 3 to 14 days. Further, after the detection of hCG is confirmed, BMP4 may be or may not be added. Examples of the suspension culture include stirring culture, stationary culture, and a combination of stirring culture and stationary culture. Further, a microcarrier can be used as necessary in the stationary culture.

Bringing Pluripotent Stem Cell into Spheroid State

The method according to the embodiment of the present invention preferably includes bringing the pluripotent stem cell into a spheroid state. Preferably, the pluripotent stem cell can be brought into a spheroid state by being subjected to three-dimensional suspension culture. The culture for bringing the pluripotent stem cell into a spheroid state may be carried out in the presence or absence of the bone morphogenetic protein BMP4; however, it is preferably carried out in the absence of BMP4 due to the reason that hCG is secreted for a long period of time. In addition, the culture for bringing the pluripotent stem cell into the spheroid state can be carried out in the presence of basic fibroblast growth factor bFGF.

The adding amount of bFGF is preferably 0.1 to 200 ng/mL and more preferably 35 to 100 ng/mL.

As the medium, a medium suitable for culturing a pluripotent stem cell is used. In a case where an iPS cell is used as the pluripotent stem cell in the step of bringing the pluripotent stem cell into a spheroid state, from the viewpoint that an iPS cell differentiates into the placenta, it is preferable to use StemFit (registered trade name) AK02N (Ajinomoto Co., Inc.); StemSure (registered trade name) and hPSC (FUJIFILM Wako Pure Chemical Corporation); mTeSR (registered trade name) 1 (Stemcell Technologies); or StemFlex (registered trade name). In addition, antibiotics such as Penicillin-Streptomycin (Gibco) can be added to these media as necessary. Further, it is possible to use a homemade medium suitable for pluripotent stem cell culture, which is prepared by appropriately adding a necessary growth factor such as FGF, the antibiotics described above, and various proteins such as HSA and BSA to a cell culture basal media such as αMEM or DMEM.

The culture period can be 1 to 10 days and preferably 1 to 4 days: however, it is not particularly limited.

The culture is preferably suspension culture, where the suspension culture means that cells are proliferated in a suspension state in the medium. The method for suspension culture are not particularly limited; however, examples thereof include stirring culture, stationary culture, and a combination of stirring culture and stationary culture. Further, a microcarrier can be used as necessary in the stationary culture. The stirring culture is a culture in a state where cells are suspended in a medium, but the cells are attached to the surface of a culture base material (a vessel). Examples of the stirring culture method include a method of carrying out culture while stirring a culture solution with a stirrer or a stirring blade and a method of carrying out culture by indirectly causing a culture solution inside a culture vessel to flow and move by driving the culture vessel itself. Examples of the former include a culture method using a spinner flask. The culture using a microcarrier is a culture in a state where cells are attached to the surface of the microcarrier. On the other hand, examples of the stationary culture method include a method of seeding an iPS cell on a U-shaped bottom 96-well plate (for example, #174925, Nunclon Sphere: Thermo Fisher Scientific, Inc.) for spheroid formation. The culture base material for spheroid formation that is used in this stationary culture is not limited as long as it has a structure in which cells do not adhere to the culture base material and further, the bottom shape is U-shaped and thus the seeded cells naturally gather at the lowermost part of the U-shape of the culture base material, where any shape such as a V-shape type, an M-shape type, or a flat surface can be used. The culture base material is not limited to a 96 well.

As the microcarrier, a microcarrier consisting of a synthetic polymer or a natural polymer can be used. Examples of the material thereof include, which are not limited thereto, polystyrene (PS), dextran which is a derivative of natural polysaccharides, collagen which is a natural protein, and a human-type recombinant protein (a genetically recombinated gelatin: WO2010/128672A1, WO2012/133610A1/product name: cellnest (registered trade name): FUJIFILM Corporation) which is a product of a gene recombinant of the partial sequence of the collagen. Such a microcarrier may be a homemade microcarrier, or a commercially available product may be purchased. In a case of being allowed to coexist with microcarriers, the spheroid or seeded cells incorporates the microcarrier into the inside to form a larger spheroid. In a spheroid consisting of only cells, nutrient components of the medium do not reach the cells in the central part, and necrosis (central necrosis) occurs. On the other hand, in a case where a microcarrier is incorporated into the inside of a spheroid having the same size as the above spheroid, cells in the central part are replaced with the microcarrier, and thus it is possible to efficiently supply oxygen and nutrients from the outside, which can prevent necrosis of the cells in the central part. The microcarrier may be porous or non-porous. A porous microcarrier is more preferable since it is expected to provide a structure more similar to the actual tissue.

The microcarrier may be or may not be coated with an adhesive substrate such as collagen or laminin; however, it is preferably coated.

In a case of culturing using a microcarrier, stationary culture is preferable.

The diameter of the microcarrier is preferably 10 to 2,500 μm and more preferably 50 to 1,000 μm. The pore diameter of the porous microcarrier is not particularly limited; however, it is preferably 5 to 500 μm.

The adding amount of the microcarrier is not limited as long as the base material has a structure in which cells do not adhere to the base material and naturally gather at the lowermost part thereof, where any shape of a V-shape type, an M-shape type, or a flat surface can be used in addition to the U-shape type. Due to not depending on the kind (the number of wells in the plate or the dish diameter) of culture base material used, the adding amount thereof is no particular limited; however, 1 to 1,000 microcarriers per spheroid is preferable, and 1 to 100 microcarriers are more preferable, from the viewpoint that a spheroid efficiently incorporated the microcarrier.

Subjecting Pluripotent Stem Cell in Spheroid State to Suspension Culture in Presence of Bone Morphogenetic Protein BMP4

In the present invention, it is preferable to subject the pluripotent stem cell in the spheroid state to suspension culture in the presence of the bone morphogenetic protein BMP4 after the pluripotent stem cell is brought into the spheroid state. In addition, it is more preferable that subjecting the pluripotent stem cell to suspension culture in the presence of the bone morphogenetic protein BMP4 is carried out in the presence of basic fibroblast growth factor bFGF.

The adding amount of BMP4 is preferably 0.1 to 1,000 ng/mL and more preferably 1 to 100 ng/mL.

The adding amount of bFGF is preferably 0.1 to 1,000 ng/mL and more preferably 1 to 100 ng/mL.

As the medium, a medium suitable for culturing a pluripotent stem cell is used as described above.

The culture period for culturing the pluripotent stem cell in the spheroid state in the presence of BMP4 is not particularly limited; however, it is preferably 2 to 20 days and more preferably 3 to 10 days. Further, after the detection of hCG is confirmed, BMP4 may be or may not be added.

As described above, the culture may be suspension culture, and the method therefor may be stirring culture or stationary culture. In addition, stationary culture can be carried out after stirring culture. The time for switching to stationary culture after stirring culture is preferably 1 to 10 days and more preferably 1 to 4 days; however, it is not particularly limited.

Subjecting the pluripotent stem cell in the spheroid state to suspension culture in the presence of the bone morphogenetic protein BMP4 may be carried out in the presence of microcarriers. As the microcarrier, the same microcarrier as described above can be used.

### One Embodiment of Present Invention

In one embodiment of the present invention, 30 mL of an iPS cell medium (for example, StemFit (registered trade name) AK02N: Ajinomoto Co., Inc.) is dispensed into a 30 mL volume spinner flask (ABLE Corporation), BMP4 is added thereto, and then iPS cells (around 1×107 cells/bottle) are seeded. BMP4 may be added to the medium at the start of culture; however, it may not be added at the start of culture and may be added to the medium around the 4th day of culture. Then, half of the medium is exchanged every 3 to 4 days; however, the addition of BMP4 is continued until about the 10th to 21st day of culture, and thereafter, only the medium is exchanged with a medium to which BMP4 is added. Around the 4th day of culture, cell aggregates (spheroids) are formed and then induced to differentiate into trophoblastic lineage cells that constitute the placenta to produce hCG, whereby a placenta-like organoid is prepared. Stirring culture may be continued in a spinner flask from the initial stage of culture; however, after the spheroid formation, the culture may be transferred to a 6-well plate for suspension culture or the like and transferred to stationary culture. The guideline for stopping the addition of BMP4 to the medium is producing hCG, and it is possible to stop the addition of BMP4 at any time after the production of hCG is started. It is also possible to continue to add BMP4 after the production of hCG is started. The culture vessel is not limited to a 6-well plate as long as it is a culture vessel for suspension culture, in which cells do not adhere, and a cell culture vessel such as a dish or a flask can be used in addition to each of the plates having 12 wells, 24 wells, and the like. In addition, as another embodiment of the spheroid formation, a U-shaped bottom 96-well plate (for example, Nunclon Sphere: Thermo Fisher Scientific, Inc.) for spheroid formation or the like can be used. The culture vessel is not limited to the U-shaped bottom 96-well plate as long as it is a suspension culture vessel in which cells do not adhere and which has such a shape that a spheroid can be formed, and it is possible to use a cell culture vessel such as a dish or a flask, having a shape of a V-shaped bottom, an M-shaped bottom, or a flat surface, in addition to each of the plates having 12 wells, 24 wells, and the like. 200 μL of an iPS cell medium (for example, StemFit (registered trade name) AK02N: Ajinomoto Co., Inc.) is dispensed into each well, BMP4 is added thereto, and then iPS cells (around 1×105 to 1×106 cells/well) are seeded. After spheroid formation, the culture may be transferred from the U-shaped bottom 96-well plate to various culture base materials such as a 6-well plate for suspension culture. Then, the culture is continued while exchanging half of the medium every 3 to 4 days. The frequency of medium exchange can be freely set within a period not exceeding 7 days.

In addition, in a case where spheroids are sampled from stirring culture with a spinner flask and transferred to a U-shaped bottom 96-well plate to transfer the culture to stationary culture, or in a case where iPS cells are directly seeded on a U-shaped bottom 96-well plate and subjected to stationary culture to form spheroids, the above-described microcarrier may be allowed to coexist.

Placenta-Like Organoid

According to the present invention, there is provided a placenta-like organoid that is produced by the method of the present invention described above. The organoid is an organized body similar to an organum or an organ, which is artificially prepared in vitro. The organoid is generally prepared by culturing a cell such as a progenitor cell or a stem cell, which contributes to organogenesis under conditions that mimic the development or regeneration processes in vivo. The placenta-like organoid is an organoid similar to the placenta and is composed of at least one or more kinds of trophoblastic lineage cells constituting the placenta in vivo, for example, a syncytiotrophoblast (ST), a cytotrophoblast (CT), an extravillous trophoblast (EVT), and trophectoderm (TE) which is a tissue of a precursor cell of these cells.

The diameter of the placenta-like organoid is preferably 50 μm to 3 cm and more preferably 500 μm to 2 cm.

The placenta-like organoid can secrete hCG for a long period of time; however, the period of secreting hCG is preferably 14 days or longer and more preferably 20 days or longer.

The placenta-like organoid that is produced by the method according to the embodiment of the present invention can preferably produce at least one (more preferably two or more and still more preferably three or more) selected from the group consisting of chorionic gonadotropin (hCG), estradiol, dehydroepiandrosterone, 11-deoxycorticosterone, progesterone, pregnenolone, and allopregnanolone. Particularly preferably, the placenta-like organoid can produce chorionic gonadotropin, estradiol, dehydroepiandrosterone, 11-deoxycorticosterone, progesterone, pregnenolone, and allopregnanolone.

In addition, the placenta-like organoid may include a syncytiotrophoblast (ST), a cytotrophoblast (CT), an extravillous trophoblast (EVT), and trophectoderm (TE) which is a tissue of a precursor cell of these cells.

A commercially available pharmaceutical product (a pregnancy examination drug) for in vitro diagnosis can be used to confirm the production of human chorionic gonadotropin hCG, which is a hormone that is produced by the placenta, and GONASTICK W (MOCHIDA PHARMACEUTICAL Co., Ltd.) is one of the above, to which an immunochromatography is applied. The pharmaceutical product for in vitro diagnosis to be used is not particularly limited as long as it is a product that detects the same hCG. About 0.5 to 1 mL of the culture supernatant is sampled, and a test strip of the pregnancy examination drug is soaked therein. Originally, examination using the pregnancy examination drug is carried out by collecting the urine of a subject and carrying out soaking in the collected urine in the urine addition part for about 3 seconds; however, the culture supernatant collected in the same manner is used for soaking the urine addition part of the test strip. After the soaking is completed, the determination surface of the test strip is placed to face upward and allowed to stand, and it is waited until the culture supernatant passes through the determination window and reaches the reaction end window. In a case where hCG is present in the culture supernatant, hCG reacts (the first reaction) with a latex particle-labeled anti-hCG antibody and moves together with the culture supernatant on the membrane of the test strip. Next, this reactant reacts (the second reaction) with an anti-hCG antibody immobilized on the membrane to form a complex of a latex particle-labeled anti-hCG antibody-hCG-immobilized anti-hCG antibody, and a blue determination line is displayed in the determination window (positive). On the other hand, in a case where hCG is not present in the culture supernatant, neither the first reaction nor the second reaction occurs, and thus a blue determination line is not displayed (negative). In addition, the reaction end sign is displayed as a pink line in the reaction end window in a case where the colorless reagent applied onto the membrane comes into contact with the culture supernatant. This examination drug is a qualitative evaluation reagent having a minimum hCG detection sensitivity of 25 IU/L. However, a blue control line corresponding to an hCG concentration of 1,000 IU/L is printed in advance on the determination window, and in a case where the hCG concentration is 1,000 IU/L or more, the determination line displays a coloration equal to or higher than that of the control line, and thus it is possible to visually check whether the hCG concentration is roughly 1,000 IU/L or more or less than 1,000 IU/L.

Further, in a case of quantifying hCG in the culture supernatant, it is possible to use the i-STAT cartridge Total β-hCG (Abbott Laboratories), which is the same pharmaceutical product for in vitro diagnosis and to which an enzyme immunoassay (an EIA method) is applied, can be used. About 17 μL of the culture supernatant is injected into a cartridge, and the cartridge is inserted into a single-purpose analyzer to start measurement. After about 10 minutes, the measurement result is automatically printed. Components involved in the reaction system in the cartridge include an anti-β-hCG mouse monoclonal antibody-alkaline phosphatase conjugate, an anti-β-hCG mouse monoclonal antibody, and sodium aminophenyl phosphate which is a substrate of alkaline phosphatase. The activity of alkaline phosphatase varies depending on the amount of hCG which is an antigen, and thus hCG can be quantified by detecting the product aminophenol.

The production of estradiol, dehydroepiandrosterone, 11-deoxycorticosterone, progesterone, pregnenolone, and allopregnanolone can be also checked with a commercially available or homemade kit using the immunochromatography in the same manner as in the checking of the production of human chorionic gonadotropin hCG, and alternatively, the checking can be carried out using a general method with which these hormones can be detected, such as liquid chromatography mass spectrometry (LC-MS) or liquid chromatography tandem mass spectrometry (LC-MS/MS). Alternatively, a highly sensitive enzyme assay method, immunoassay method, chemiluminescence method, or fluorescence emission method may be used with a commercially available or homemade drug for in vitro diagnosis using whole blood, plasma, serum, urine, or another specimen, which is generally used for detecting a trace amount of hormones present in the living body. Examples of such an assay method include ELISA (one kind of the enzyme immunoassay (EIA)), an electrochemiluminescence (ECL) method, and a radioimmunoassay method (RIA) method.

Use Application of Placenta-Like Organoid and Kit

The present invention further relates to a production or test kit including the placenta-like organoid of the present invention. The kit according to the embodiment of the present invention can be used for producing a reproductive hormone, evaluating toxicity or safety of a test substance, or analyzing an infection mechanism of a virus.

As described above, the placenta-like organoid according to the embodiment of the present invention is capable of producing at least one reproductive hormone selected from the group consisting of human chorionic gonadotropin, estradiol, dehydroepiandrosterone, 11-deoxycorticosterone, progesterone, pregnenolone, and allopregnanolone. As a result, in a case where the placenta-like organoid according to the embodiment of the present invention is cultured. The above-described reproductive hormones can be produced.

The placenta-like organoid according to the embodiment of the present invention can be used to evaluate the toxicity or safety of a test substance. The placenta-like organoid according to the embodiment of the present invention is useful as a drug evaluation model in the pregnancy period. The placenta-like organoid according to the embodiment of the present invention has a three-dimensional structure (a spacial structure) and thus can be highly organized and made to a chip. The element having the function of the organ, which is constructed on a chip, is also referred to as an Organ on a chip or a biofunctional chip. The placenta-like organoid according to the embodiment of the present invention can be used to construct an Organ on a chip or a biofunctional chip. Furthermore, in a case where the placenta-like organoid according to the embodiment of the present invention is connected to another organ-like organoid, it is possible to evaluate the effects of drugs, alcohol, food, and the like on each tissue in the human development process and fetal development process.

In a case of evaluating the toxicity or safety of a test substance using the placenta-like organoid according to the embodiment of the present invention, it is possible to bring a test substance into contact with the placenta-like organoid according to the embodiment of the present invention. Specifically, it is possible to add a test substance to the culture medium containing the placenta-like organoid according to the embodiment of the present invention and culture the placenta-like organoid in the presence of the test substance. The culture period is not particularly limited; however, it is generally 1 hour to 30 days. However, the culture period can be extended as necessary.

As the test substance, organic compounds or inorganic compounds having various molecular weights can be used. Examples of the organic compound include, which are not particularly limited, a nucleic acid, a peptide, a protein, a lipid (a simple lipid, a complex lipid (a phosphoglyceride, a sphingolipid, a glycosyl glyceride, a cerebroside, or the like), a prostaglandin, an isoprenoid, a terpene, a steroid, a polyphenol, catechin, and vitamins. It may be an existing component or candidate component such as a pharmaceutical product, a nutritional food, a food additive, a pesticide, or perfumery (a cosmetic). A plant extract, a cell extract, a culture supernatant or the like may be used as the test substance. In a case where two or more test substances are added at the same time, the interaction, the synergism, or the like between the test substances can be investigated. The test substance may be of natural origin or may be obtained by synthesis. In a case where a test substance obtained by synthesis is used, an efficient assay system can be constructed using, for example, a combinatorial synthesis method.

Since whether or not a test substance permeates the placenta greatly affects the exhibition of developmental toxicity to the fetus due to the test substance, it is possible to evaluate the toxicity or safety of the test substance by evaluating the human placental permeability of the test substance.

For example, the placenta-like organoid can be cultured on a semi-permeable membrane (a porous membrane), and a test substance that has permeated the placenta-like organoid can be quantified. The quantification of the test substance can be performed by a measurement method such as mass spectrometry, liquid chromatography, an immunological method (for example, a fluorescence immunoassay method (an FIA method), or an enzyme immunoassay (an EIA method)) depending on the kind of test substance. The human placental permeability of the test substance can be evaluated based on the quantification result (the amount of the test substance that has permeated the placenta-like organoid) and the using amount of the test substance (typically, the amount added to the medium). Further, the presence or absence of human placental permeability of the test substance can be qualitatively evaluated. In that case, it is possible to use a commercially available or homemade drug for in vitro diagnosis or a homemade test kit similar thereto, with which the test substance is capable of being detected by the immunochromatography or other principles, such as those used for the detection of hCG.

In the maternal-fetal transmission of a pathogen such as a virus, it is known that after the mother is infected with a pathogen, the fetus is infected with the pathogen via the placenta. Examples of the pathogen include a virus, a bacterium, and a fungus; however, the pathogen is not particularly limited.

In order to analyze the infection mechanism of a pathogen such as a virus, a human fibroblast cell line or the like is cultured in a medium to which FBS, antibiotics, and the like have been added, and the cells are infected with a pathogen such as a virus strain to cause the pathogen to proliferate to a certain quantity. This pathogen is added to the placental-like organoid culture solution to carry out an infection experiment.

As another use application, an extract having various physiological activities, which is obtained from the placenta-like organoid, can be used in cosmetics, foods, pharmaceutical products, and the like.

The present invention will be further specifically described with reference to Examples; however, the present invention is not limited by Examples.

### EXAMPLES

In Comparative Examples and Examples below, the production of placental hormone hCG was checked by the qualitative evaluation using GONASTICK W (MOCHIDA PHARMACEUTICAL Co., Ltd.) and the quantitative evaluation using i-STAT cartridge Total β-hCG (Abbott Laboratories).

In GONASTICK W, about 0.5 to 1 mL of the culture supernatant was sampled, and a test strip was soaked therein. After the soaking was completed, the determination surface of the test strip was placed to face upward and allowed to stand until the culture supernatant passed through the determination window and reached the reaction end window. The reaction end sign is displayed as a pink line in the reaction end window. The detection sensitivity of the present examination reagent is 25 IU/L, and a blue control line corresponding to an hCG concentration of 1,000 IU/L is printed in advance on the determination window, which can be visually compared with the determination line.

In a case where a line equal to or more blue-colored than the control line was visually observed on the determination line, it was judged as positive (+).

In a case where the line was not visually observed on the determination line, it was determined to be negative (−).

In a case where a line that was less blue-colored than the control line but was visible was visually observed on the determination line, it was determined to be (+/−).

In the i-STAT cartridge Total β-hCG, about 17 μL of the culture supernatant was sampled, injected into the reagent cartridge, and inserted into the single-purpose analyzer. The value displayed after about 10 minutes was used as the quantification value of hCG.

As the iPS cells used in Comparative Examples and Examples below, those prepared from a commercially available human adipose-derived hepatocyte ADSC (PT-5006: Lonza) according to the basic iPS cell preparation method as described above were used. In addition, as the iPS cells used in Comparative Examples and Examples, those subcultured in StemFlex (Thermo Fisher Scientific, Inc.) were used. In addition, in the use in Comparative Examples and Examples, the medium of iPS cells was changed to StemFit (Ajinomoto Co., Inc.) or StemSure (FUJIFILM Wako Pure Chemical Corporation) from the viewpoint of iPS cell differentiation.

### Comparative Example 1: Two-Dimensional Adhesion Culture

Laminin (iMatrix-511: Nippi. Inc.) was diluted with Dulbecco's phosphate buffered saline (DPBS), 1 mL thereof per well was dispensed into a 6-well plate (Falcon TC: #353046), and the plate was allowed to stand in a 37° C. incubator for 1 hour to be coated with laminin. 2 mL of an iPS cell medium (StemFit (registered trade name) AK02N: Ajinomoto Co., Inc.) to which 2 μL of a solution containing 50 μg/mL BMP4 (R&D Systems Inc.) had been added was dispensed into the laminin-coated plate, and iPS cells (4×104 cells/well) were seeded therein. The culture was continued while appropriately changing the medium, and the hCG contained in the culture supernatant was detected using a drug for in vitro diagnosis for the pregnancy test, GONASTICK W (MOCHIDA PHARMACEUTICAL Co., Ltd.), whereby the differentiation induction into a placental tissue was checked. As a negative control, the same culture was carried out using a medium to which BMP4 had not been added. The hCG was started to be produced on the 7th day of culture; however, the hCG production stopped around the 18th day (Table 1).

TABLE 1

| Medium | BMP4 | Days | 7 | 11 | 15 | 18 | 21 |
|---|---|---|---|---|---|---|---|
| StemFitR | Absent | hCG | − | − | − | − | − |
| | | production | | | | | |
| StemFitR | Present | hCG | + | + | + | +/− | − |
| | | production | | | | | |

### Example 1

Three-Dimensional Suspension Culture

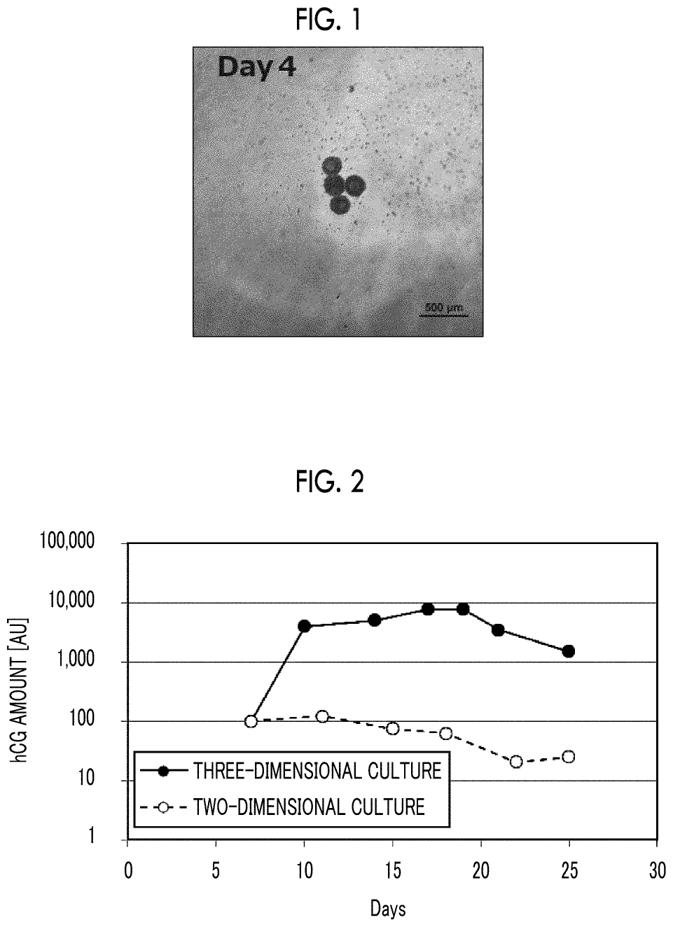

30 mL of an iPS cell medium (StemFit (registered trade name) AK02N: Ajinomoto Co., Inc.) was dispensed into a 30 mL volume spinner flask (ABLE Corporation), and then iPS cells (1.6×107 cells/mL) were seeded. BMP4 was not added at the start of culture, but 30 μL of a solution containing 50 μg/mL BMP4 (R&D Systems Inc.) was added on the 4th day of culture so that the final concentration of BMP4 in the medium was to be 50 ng/mL. In a case where BMP4 was added to Example 2 and subsequent Examples, the same concentration was used. After the start of culture, the addition of BMP4 was continued until the 18th day of culture while exchanging half of the medium every 3 to 4 days, and thereafter, only the medium was exchanged without adding BMP4. Cell aggregates (spheroids) were formed by the 4th day of culture (FIG. 1). The stirring culture was continued in a spinner flask throughout the culture period. Although the hCG production was not confirmed until the 11th day of culture, the hCG production was confirmed after the 15th day of culture, and it was confirmed that the hCG production was continued even after the 60th day of culture (Table 2).

TABLE 2

| Days | 15 | 39 | 53 | 67 |
|---|---|---|---|---|
| hCG | + | + | + | + |
| production | | | | |

### Example 2

Three-Dimensional Suspension Culture

30 mL of an iPS cell medium (StemFit (registered trade name) AK02N: Ajinomoto Co., Inc.) was dispensed into a 30 mL volume spinner flask (ABLE Corporation), and then iPS cells (1.1×107 cells/bottle) were seeded. The comparison was carried out between the level 1 in which BMP4 was not added at the start of culture but added on the 4th day of culture and the level 2 in which BMVP was added from the start of culture. BMP4 was added so that the concentration in the medium was 50 ng/mL in both the level 1 and the level 2. After the start of culture, half of the medium was exchanged every 3 to 4 days, and after the 18th day of culture, the medium was exchanged for both the level 1 and the level 2 with a medium to which BMP4 had not been added. By the 4th day of culture, cell aggregates (spheroids) were formed. Regarding the level 1 and the level 2, stirring culture was continued in a spinner flask from the initial stage of culture, and the hCG production was checked. In a case where BMP4 is added from the start of culture (the level 2), the hCG production is started earlier; however, the hCG production also stops earlier. On the other hand, in a case where BMP4 is added after the 4th day of culture (the level 1), the hCG production is slightly delayed; however, it continued for a long time.

TABLE 3

| | | BMP | | | | | | | | | |
|---|---|---|---|---|---|---|---|---|---|---|---|
| | | addition | | | | | | | | | |
| | | Day | Day | HCG production (Day) | | | | | | | |
| Level | Medium | 0~ | 4~ | 8 | 11 | 14 | 18 | 21 | 25 | 28 | 32 |
| 1 | StemFitR | No | Yes | − | − | +/− | + | + | + | + | + |
| 2 | StemFitR | Yes | Yes | + | + | + | + | + | +/− | − | − |

### Example 3

Simultaneous Comparison of Two-Dimensional Adhesion Culture and Three-Dimensional Suspension Culture

Two-dimensional adhesion culture was carried out by the following method. Laminin (iMatrix-511: Nippi. Inc.) was diluted with Dulbecco's phosphate buffered saline (DPBS), 1 mL thereof per well was dispensed into a 6-well plate (Falcon TC: #353046), and the plate was allowed to stand in a 37° C. incubator for 1 hour to be coated with laminin. 2 mL of an iPS cell medium (StemFit (registered trade name) AK02N: Ajinomoto Co., Inc.) was dispensed into the laminin-coated plate in a state where a BMP4 solution was not added, iPS cells (4×104 cells/well) were seeded therein, the culture was continued while appropriately changing the medium, and from the 4th day of culture, 2 mL of an iPS cell culture medium (StemFit (registered trade name) AK02N: Ajinomoto Co., Inc.) to which 2 μL of a solution containing 50 μg/mL BMP4 (R&D Systems Inc.) solution had been added so that the BMP4 concentration was to be 50 ng/mL was used at the time of the medium exchange.

On the other hand, three-dimensional suspension culture was started at the same time using the same iPS cells used in the two-dimensional culture adhesion culture. 30 mL of an iPS cell medium (StemFit (registered trade name) AK02N: Ajinomoto Co., Inc.) was dispensed into a 30 mL volume spinner flask (ABLE Corporation), and then iPS cells (1.3×106 cells/mL) were seeded. In the same manner as in the two-dimensional adhesion culture, BMP4 was not added at the start of culture, but a medium to which a solution containing 50 μg/mL BMP4 (R&D Systems Inc.) was added at the same proportion as that used in the two-dimensional adhesion culture was used after the 4th day of culture. After the start of culture, the addition of BMP4 to the medium was continued until the 14th day of culture while the medium was exchanged every 3 to 4 days, and thereafter, a medium to which BMP4 had not been added was used. In three-dimensional suspension culture, stirring culture was continued in a spinner flask throughout the culture period. While continuing the two-dimensional adhesion culture and the three-dimensional suspension culture, the culture supernatant was appropriately sampled after the 7th day of culture, and the amount of hCG produced was quantified using the i-STAT cartridge Total β-hCG. The hCG quantification results are shown in Table 4. Since there are differences in the number of cells that can be seeded at the start of culture and the adding amount of medium between the two-dimensional culture and the three-dimensional culture, there are differences in the hCG concentration and the total hCG production in the culture supernatant, and thus it is not possible to simply compare them. For this reason, the hCG quantification value on the 7th day of culture after the start of differentiation induction into the placenta was set to 100, and the ratio of the hCG quantification value on the subsequent culture measurement day to the hCG quantification value on the 7th day of culture was calculated. That is, in a case where the hCG quantification value on the 7th day is set to 100 in each of the two-dimensional and three-dimensional culture forms, the subsequent changes after the 7th day in the hCG production amount are shown in FIG. 2. It was confirmed that in the two-dimensional culture, the decrease in hCG production amount is particularly remarkable after the 15th day of culture, whereas in the three-dimensional culture, the hCG continues to be produced after the 15th day, and even on the 25th day of culture, 10 times or more of the hCG production is maintained as compared with the 7th day of culture.

TABLE 4

| Two-dimensional culture | | | Three-dimensional culture | | |
|---|---|---|---|---|---|
| | hCG | hCG* | | hCG | hCG* |
| Days | production | production | Days | production | production |
| [days] | [IU] | [AU] | [days] | [IU] | [AU] |
| 7 | 6.6 | 100 | 7 | 1.6 | 100 |
| 11 | 8.0 | 120 | 10 | 61 | 3,936 |
| 15 | 5.0 | 75 | 14 | 79 | 5,044 |
| 18 | 4.1 | 62 | 17 | 120 | 7,692 |
| 22 | 1.4 | 20 | 19 | 120 | 7,692 |
| 25 | 1.6 | 25 | 21 | 54 | 3,465 |
| — | — | — | 25 | 23 | 1,498 |

*(hCG production on each of culture days/hCG production amount on 7th day) × 100

### Example 4

Three-Dimensional Suspension Culture

In a case where iPS cells were seeded on a U-shaped bottom 96-well plate (#174925, Nunclon Sphere: Thermo Fisher Scientific, Inc.) for spheroid formation to be subjected to stationary culture, microcarriers consisting of a natural polymer or a synthetic polymer were allowed to coexist.

The following three kinds of levels 3 to 5 were used. The culture was carried out using a total of four levels including a level 6 as a negative control, in which only spheroids were seeded without adding microcarriers.

Level 3: Porous microspheres using, as a material, collagen which is a natural protein, and a human-type recombinant protein (a genetically recombinated gelatin: WO2010/128672A1, WO2012/133610A1/product name: cellnest: FUJIFILM Corporation) which is a product of a gene recombinant of the partial sequence of the collagen; microsphere (MS)

Level 4: Microcarriers (Cytodex 3, 17-0485, General Electric Company) having a collagen layer chemically bonded to the surface of crosslinked dextran, which is a derivative of a natural polysaccharide; Cytodex 3

Level 5: Microcarriers obtained by coating the surface of polystyrene (PS) with collagen (3786, Corning Inc.); collagen-coated microcarrier (MC)

Level 6: Control (spheroids only)

200 μL of an iPS cell medium (StemFit (registered trade name) AK02N: Ajinomoto Co., Inc.) to which BMP4 (R&D Systems Inc.) had not been added was dispensed into each well of the U-shaped bottom 96-well plate for spheroid formation without laminin coating. The above-described level 3 to 5 microcarriers containing the negative control (the level 6) were added to the well in a range of 3 to 200 microcarriers, and iPS cells (4×105 cells/well) were seeded. In a case where the cells were allowed to coexist with the microcarriers, the seeded cells incorporated the microcarriers into the inside to form spheroids. After the 4th day of culture on which the spheroids were formed, the medium exchange was started with an iPS cell medium to which BMP4 had been added, and after the 10th day of culture, the medium was changed again to an iPS medium to which BMP4 had not been added. On the 17th day of culture, the spheroids were transferred from the 96-well plate to a 6-well plate for suspension culture (#3471, 6-well Flat Button, Ultra-Low Attachment Surface: Corning Inc.) into which 2 mL of the medium had been dispensed in advance. Then, the culture was continued while exchanging half of the medium every 3 to 4 days. As a result of checking the hCG production on the 25th day of culture using GONASTICK W, hCG was produced with all the levels 3 to 6; however, the color development of the level 3 of the porous microspheres was strongest.

TABLE 5

| | hCG production |
|---|---|
| Level | (Day 25) |
| 3 | + |
| 4 | + |
| 5 | + |
| 6 | + |

### Example 5

Three-Dimensional Suspension Culture

30 mL of an iPS cell medium (StemFit (registered trade name) AK02N: Ajinomoto Co., Inc.) to which BMP4 (R&D Systems Inc.) had not been added was dispensed into a 30 mL volume spinner flask (ABLE Corporation), iPS cells (around 1.1×107 cells/bottle) were seeded, and then stirring culture was started. On the 4th day of culture, 200 μL of an iPS cell medium (StemFit (registered trade name) AK02N: Ajinomoto Co., Inc.) to which BMP4 had been added was dispensed into each well of the U-shaped bottom 96-well plate (#174925, Nunclon Sphera: Thermo Fisher Scientific, Inc.). The same microcarriers of the level 3 and the level 5 as in Example 3, including the negative control (the level 6), were added to the well, the formed spheroids were subsequently sampled from the spinner flask, and about 2 to 20 spheroids were dispensed and subjected to stationary culture. The level 6, for which only spheroids were added but microcarriers were not added, were compared with the level 3 and the level 5, for which both microcarriers and spheroids were added.

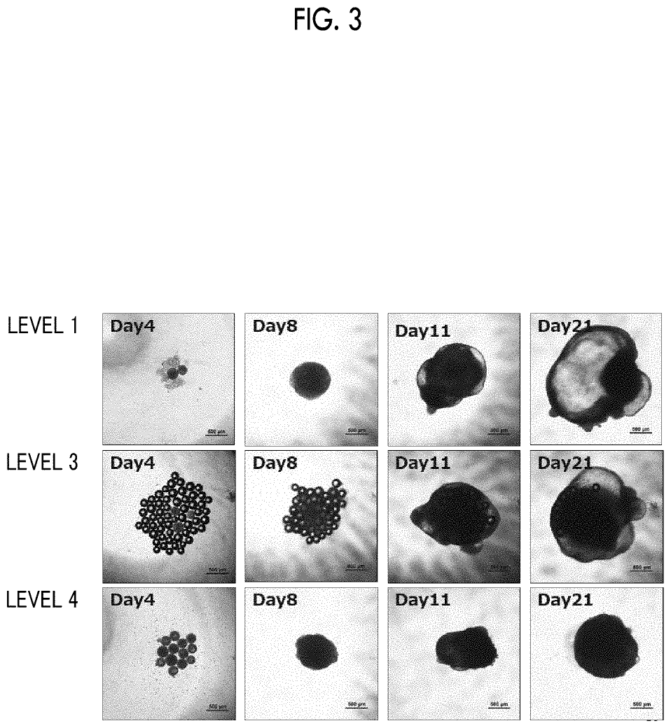

In a case of being allowed to coexist with microcarriers, the spheroids incorporated the microcarrier into the inside during the culture process to form a larger spheroid. At the timing of the 4th day of culture, in a case where two spheroids and three spheroids were added for the level 3 and the level 5, respectively, while 13 spheroids were added for the level 6, to the respective wells of the U-shaped bottom 96-well plate, at the stage of the 21st day of culture, the particle size with the levels 3 and the level 5 was larger than that with the level 6. As a clear difference was observed, in the spheroid consisting of only cells, nutrient components of the medium do not reach the cells in the central part, and necrosis (central necrosis) occurs. On the other hand, in a case where the microcarrier is incorporated into the inside of the spheroid having the same size as the above spheroid, cells in the central part are replaced with the microcarrier, and thus it is possible to prevent necrosis of the cells in the central part. As shown in FIG. 3, a lumen-like structure is observed in a case where the levels of MS (the level 3) and MC (the level 5) are added, whereas the cells remain tightly packed in a case of the level 6. As a result, it is conceivable that the addition of microcarriers provides a higher organized structure. Further, the microcarrier is incorporated into the inside of the spheroid regardless of whether it is porous or not porous; however, it is expected that a porous microcarrier provides a structure that is more similar to the actual tissue.

### Example 6

200 μL of an iPS cell medium (StemFit (registered trade name) AK02N: Ajinomoto Co., Inc.) to which BMP4 (R&D Systems Inc.) had been added to 50 ng/mL was dispensed into each well of the U-shaped bottom 96-well plate for spheroid formation, and iPS cells (1×104 cells/well) were seeded therein. The culture supernatant after culturing, for 7 days, the placental-like organoid prepared in this manner was sampled, and subjected to the checking by a liquid chromatography tandem mass spectrometer (LC-MS/MS) whether the culture supernatant contained a placenta-derived hormone other than hCG, that is, whether this organoid produced a placenta-derived hormone other than hCG. As the negative control, the chromatogram of this culture supernatant was compared with that of the iPS cell medium (StemFit (registered trade name) AK02N: Ajinomoto Co., Inc.) alone, and qualitative evaluation was performed from the obtained peaks.

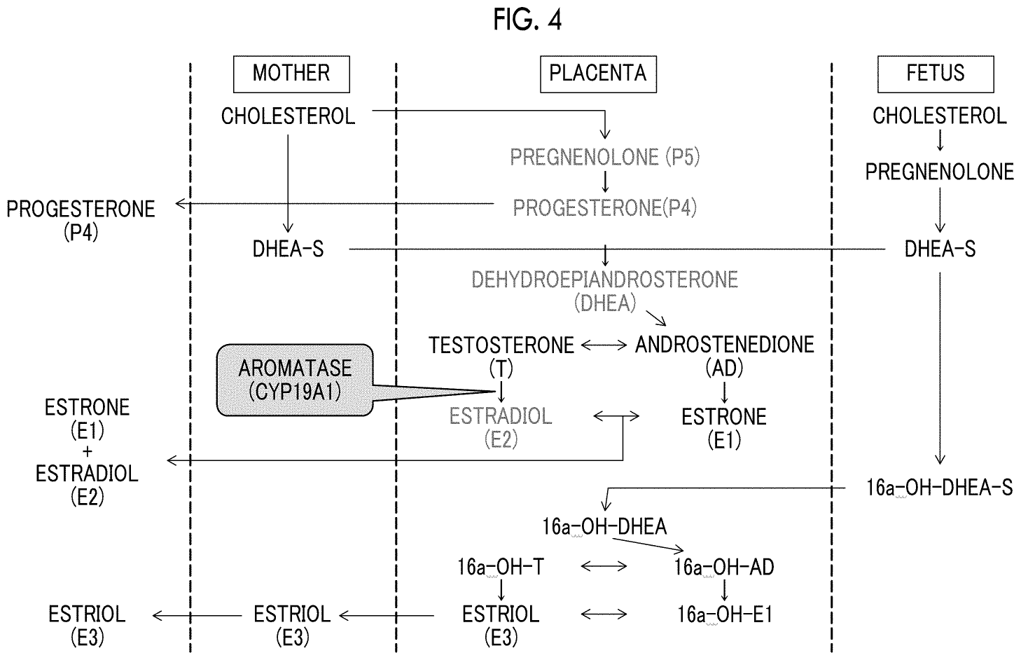

As a result of the above, the hormones shown in Table 6 were detected. All of these are known as steroid hormones produced by the placenta (FIG. 4).

TABLE 6

| Hormone | Culture supernatant | | Medium | |
|---|---|---|---|---|
| Estradiol (E2) | + | 2.51 | − | 0.11 |
| Dehydroepiandrosterone (DHEA) | + | 1.57 | − | 1.28 |
| 11-deoxycorticosterone (DOC) | + | 2.70 | − | 0.02 |
| Progesterone (P4) | + | 2674 | − | 0.10 |
| Pregnenolone (P5) | + | 3805 | − | 0.44 |
| Allopregnanolone (AP) | + | 12.31 | − | 0.68 |

+: Regarding an area (Ssample) of a peak corresponding to the position of each hormone in the chromatogram obtained from each of the samples of the culture supernatant and the medium and an area (Sstd) of a peak of a standard solution containing only the hormone of a known concentration (1 pg/mL), Ssample/Sstd was calculated, and a case where the calculated value was more than 1.5 was determined to be (+).
−: A case where a value determined according to the above expression was less than 1.5 was determined to be (−).

### Example 7

Checking of Viral Infection to Placental-Like Organoid

A human fibroblast cell line MRC-5 (purchased from ATCC, ATCC CCL-171) was cultured in a DMEM medium containing 10% FBS (Invitrogen) and 1% antibiotics (penicillin/streptomycin, 10,000 U/mL: Thermo Fisher Scientific, Inc.). Human cytomegalovirus (HCMV) AD-169 strain (purchased from ATCC, ATCC VR-538) was added to the culture solution of the MRC-5 cells so that the cells were infected. After confirming that most of the MRC-5 cells were dead 10 days after the infection, the culture supernatant in which the proliferated virus particles were released to the outside of the MRC-5 cells was collected, and the cell debris contained in the culture supernatant was removed by centrifugation. The remaining culture supernatant was passed through a 0.45 μm filter to obtain an AD-169 solution. This AD-169 solution was added to the placental-like organoid culture solution on the 16th day of culture, and an experiment of HCMV infection of the placental-like organoid was performed. The placental-like organoid used for HCMV infection in present Example was prepared in the same manner as in Example 5 by culturing for 16 days. Total RNA was extracted from the infected placental-like organoid using a TRIZOL reagent (Invitrogen, #15596026). First, homogenization was performed by adding 1 mL of the TRIZOL reagent per 5 to 10×106 cells. Next, 0.2 mL of chloroform was added, and the resultant mixture was allowed to stand at room temperature for about 2 to 3 minutes and then centrifuged (12,000 rpm, 15 minutes, 4° C.). The upper layer (the aqueous layer) was taken into another container, 0.5 mL of isopropanol was added thereto and allowed to stand at room temperature for about 5 to 10 minutes. After adding 1 mL of 75% ethanol to the precipitated RNA, centrifugation (7,500 rpm, 5 minutes, 4° C.) and then washing was carried out to obtain total RNA. The RNA precipitate was air dried for about 5 to 10 minutes (the details of the operation were carried out according to the attached document of the TRIZOL reagent).

From the extracted total RNA, cDNA was synthesized using a PrimeScript II 1st strand cDNA Synthesis Kit (Takara Bio Inc., #6210A). First, 1 μL of an oligo dT primer, 1 μL of dNTP Mixture, 5 μg or less of the extracted total RNA in terms of RNA amount, and RNA free dH2O (added so that the total liquid volume is 10 μL) were mixed in a microtube, the resultant mixture was kept warm at 65° C. for 5 minutes and then rapidly cooled on ice. To this solution, 4 μL of 5× PrimerScript II Buffer, 0.5 μL of RNase Inhibitor, 1 μL of PrimeScript II RTase, and RNA free dH2O (added so that the total amount of the solution was 20 μL) were added and gently stirred. Then, the reaction was carried out promptly at 30° C./10 minutes and subsequently at 42° C./30 to 60 minutes. The enzyme was inactivated by keeping the temperature at 95° C. for 5 minutes and then cooled on ice to prepare a cDNA solution.

Using the synthesized cDNA, RT-PCR and electrophoresis were performed on each of an immediate early 2 (IE2) gene and a TATA-binding protein (TBT: a basal transcription factor that binds to a DNA sequence called a TATA box) gene. The PCR reaction solution, the reaction conditions, and the electrophoresis conditions for each gene were as follows. In addition, a placental-like organoid which had not been infected with HCMV was used as MOCK cells in the electrophoresis experiment. This is a placental-like organoid cultured only in the medium used for the culture of placental-like organoid, to which the solution containing HCMV AD-169 virus particles is added.

(1) IE2 Gene
PCR Reaction Solution
cDNA: 1 μL
Forward primer: 0.5 μL
Reverse primer: 0.5 μL
H2O: 10 μL
2× Buffer: 12.5 μL
Tks Gflex DNA Polymerase: 0.5 μL
(Takara Bio Inc., #R060A)

PCR primer sequence
Forward:
(SEQ ID NO: 1)
CGCAAGCTTGCCGCCACCATGGAGTCCTCTGCCAAGAGAAAG
Reverse:
(SEQ ID NO: 2)
CGCGGATCCCTGAGACTTGTTCCTCAGGTCCTG

PCR Reaction Conditions
94° C., 1 min.
Hereinafter, 32 cycles
98° C., 10 sec.
55° C., 15 sec.
68° C., 1 min.
Finally
68° C., 7 min.
The prepared PCR reaction product is stored at 4° C.

Electrophoresis Conditions
1% agarose gel

(2) TBP Gene
PCR Reaction Solution
cDNA: 1 μL
Forward primer: 0.5 μL
Reverse primer: 0.5 μL
H2O: 10 μL
2× Buffer: 12.5 μL
Tks Gflex DNA Polymerase: 0.5 μL
(Takara Bio Inc., #R060A)

PCR primer sequence
Forward:
(SEQ ID NO: 3)
TTCGGAGAGTTCTGGGATTGTA
Reverse:
(SEQ ID NO: 4)
TGGACTGTTCTTCACTCTTGGC

PCR Reaction Conditions
94° C., 1 min.
Hereinafter, 32 cycles
98° C., 10 sec.
55° C., 15 sec.
68° C., 15 sec.
Finally
68° C., 7 min.
The prepared PCR reaction product is stored at 4° C.

Electrophoresis Conditions
2% agarose gel

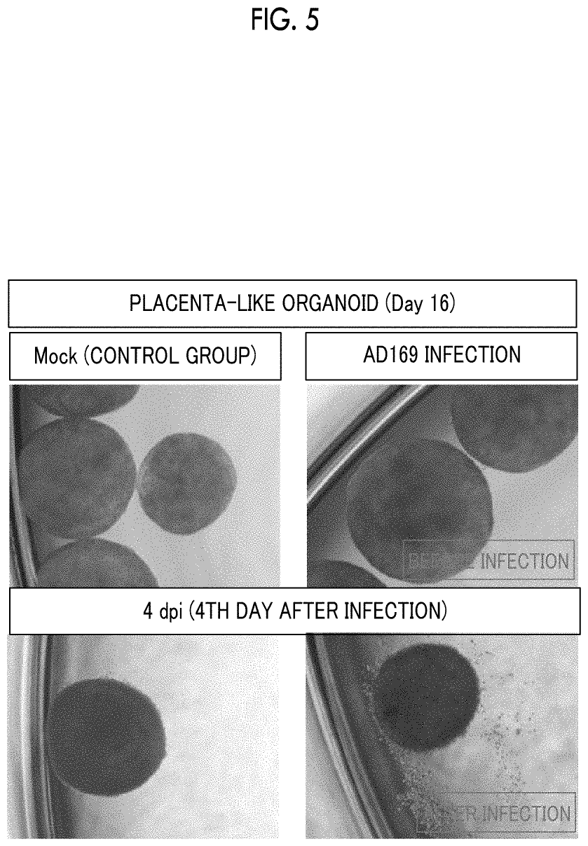

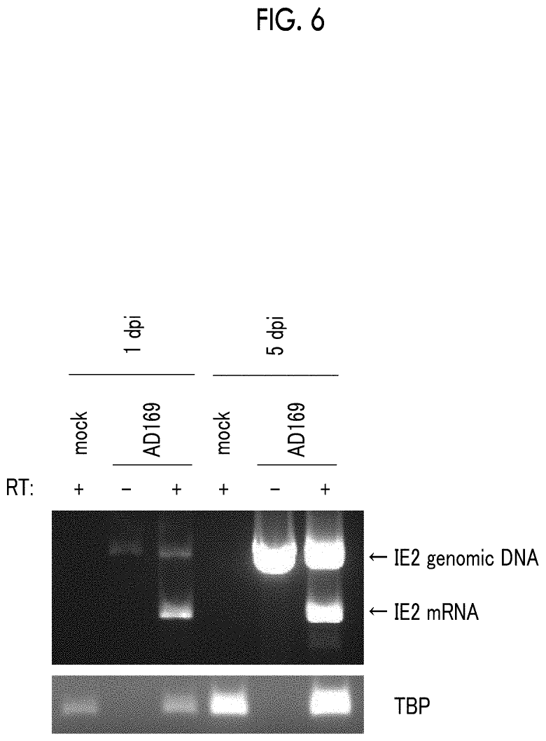

The results of comparing the appearances of placental-like organoids before infection and on the 4th day after infection in a case where the placental-like organoid has been infected with human cytomegalovirus (HCMV) AD-169 strain are shown in FIG. 5. In addition, results of checking, by electrophoresis, mRNA of the IE2 gene of which the expression is increased in the early stage of viral infection, on the 1st day after infection (1 day post infection (dpi)) and the 5th day after infection (5 dpi), in a case where the placental-like organoid is infected with human cytomegalovirus (HCMV) AD-169 strain are shown in FIG. 6. HCMV is a double-stranded DNA virus as described, for example, in Modern Media Vol. 57, No. 3, 2011, 79-85, “Cytomegalovirus infectious disease during perinatal period”, and after the cell invasion, a viral protein VP16 and a cellular transcriptional regulation factor forms a complex that induces the expression of immediate early (IE) genes. Among the IE genes, the IE1 and IE2 gene products regulate the expression of subsequent early (E: early or delayed early) and late (L: late) genes. In present Example, the genomic DNA of the AD-169 strain with which the placental-like organoid was infected was detected regardless of the presence or absence of the reverse transcriptase (RT); however, separately from that, a band that was not detected in a case where the RT was absent (RT (−)) but was detected only in a case where the RT was present (RT (+)), that is, the mRNA of the IE2 gene expressed in the cells of the organoid by the infection of the placental-like organoid with the AD-169 strain was detected. On the other hand, the reverse primer of the TBP gene is designed to be positioned across two exons. As a result, this TBP primer set detects only the cDNA synthesized from the TBP mRNA, whereas genomic DNA is not detected. In a case where the RT was absent (RT (−)), no band was detected in the PCR product since cDNA derived from the TBP mRNA was not synthesized.

According to the method according to the embodiment of the present invention, it is possible to produce a placenta-like organoid. In addition, foods or pharmaceutical products ingested by a pregnant woman are incorporated into the fetal blood in a case where they pass through the placenta. In a case where the placenta-like organoid that is produced by the method according to the embodiment of the present invention is used, it is possible to evaluate what substance passes through the placenta and how the passed-through substance affects the fetus, and further, it is possible to elucidate the infection mechanism of a virus and the like that is transmitted via the placenta.

What is claimed is:

1. A method of producing a placenta-like organoid, comprising subjecting a pluripotent stem cell to suspension culture in a presence of a bone morphogenetic protein BMP4.

2. The method according to claim 1, further comprising bringing the pluripotent stem cell into a spheroid state.

3. The method according to claim 2, further comprising subjecting the pluripotent stem cell in the spheroid state to suspension culture in a presence of the bone morphogenetic protein BMP4 after the pluripotent stem cell is brought into the spheroid state.

4. The method according to claim 2,
wherein the bringing of the pluripotent stem cell into the spheroid state is carried out in a presence of basic fibroblast growth factor bFGF.

5. The method according to claim 2,
wherein the bringing of the pluripotent stem cell into the spheroid state is carried out in an absence of the bone morphogenetic protein BMP4.

6. The method according to claim 1,
wherein the subjecting of the pluripotent stem cell to the suspension culture in the presence of the bone morphogenetic protein BMP4 is carried out in a presence of basic fibroblast growth factor bFGF.

7. The method according to claim 1,
wherein at least one of the bringing of the pluripotent stem cell into a spheroid state or the subjecting of the pluripotent stem cell to the suspension culture in the presence of the bone morphogenetic protein BMP4 is carried out in a presence of a microcarrier.

8. The method according to claim 7,
wherein the microcarrier is porous.

9. The method according to claim 1,
wherein the pluripotent stem cell is an embryonic stem cell, an embryonic germ cell, or an induced pluripotent stem cell.

10. The method according to claim 1,
wherein the placenta-like organoid is capable of producing at least one selected from the group consisting of chorionic gonadotropin, estradiol, dehydroepiandrosterone, 11-deoxycorticosterone, progesterone, pregnenolone, and allopregnanolone.

11. A placenta-like organoid that is produced by the method according to claim 1.

12. A production or test kit comprising the placenta-like organoid according to claim 11.

13. The production or test kit according to claim 12,
wherein the kit is used for producing a reproductive hormone, evaluating toxicity or safety of a test substance, or analyzing an infection mechanism of a pathogen.


SEQUENCE LISTING (4 sequences)

SEQ ID NO: 1 - DNA, 42 bp, Artificial Sequence
  Primer
cgcaagcttgccgccaccatggagtcctctgccaagagaaag

SEQ ID NO: 2 - DNA, 33 bp, Artificial Sequence
  Primer
cgcggatccctgagacttgttcctcaggtcctg

SEQ ID NO: 3 - DNA, 22 bp, Artificial Sequence
  Primer
ttcggagagttctgggattgta

SEQ ID NO: 4 - DNA, 22 bp, Artificial Sequence
  Primer
tggactgttcttcactcttggc